(19) United States
(12) Patent Application Publication
(10) Pub. No.: US 2026/0060911 A1
(43) Pub. Date: Mar. 5, 2026

(54) ESTER OF TRIFAROTENE

(71) Applicant: Actera Ingredients, Inc., Newtown, PA (US)

(72) Inventor: Daniel Winn, Princeton, NJ (US)

(73) Assignee: Actera Ingredients, Inc., Newtown, PA (US)

(21) Appl. No.: 19/383,146

(22) Filed: Nov. 7, 2025

Related U.S. Application Data
(63) Continuation of application No. PCT/US2024/044362, filed on Aug. 29, 2024.
(60) Provisional application No. 63/536,262, filed on Sep. 1, 2023.

(51) Int. Cl. A61K 8/49; A61K 8/06; A61K 8/19; A61K 8/31; A61K 8/34; A61K 8/365; A61K 8/37; A61K 8/64; A61K 8/73; A61K 8/92; A61Q 19/08; C07D 295/155

(52) U.S. Cl. CPC A61K 8/4913; A61K 8/062; A61K 8/19; A61K 8/31; A61K 8/345; A61K 8/365; A61K 8/37; A61K 8/645; A61K 8/73; A61K 8/922; A61Q 19/08; C07D 295/155

(57) ABSTRACT

Certain trifarotene fatty acid esters and external, topical skin care compositions containing one or more such compounds are described. In some embodiments, upon hydrolysis in the skin, shorter chain trifarotene fatty acid esters release short chain fatty acids such as octanoic acid and lauric acid, which are known to contribute to penetration enhancement in topical skin delivery. Methods of topically applying the external compositions to the skin of a subject to inhibit or reduce signs of photoaging or acne are also described.

### STATEMENT OF RELATED CASES

This application is a continuation of International Application No. PCT/US2024/044362, filed Aug. 29, 2024, and claims priority to U.S. Provisional Application No. 63/536,262, filed Sep. 1, 2023, the entire contents of which are incorporated herein by reference.

### FIELD OF THE INVENTION

The present invention relates to trifarotene fatty acid esters and various external compositions containing one or more trifarotene fatty acid esters, methods of using these compositions, and processes for preparing these compositions. The external compositions are applied to skin where upon hydrolysis in the skin, the shorter chain esters release short chain fatty acids such as octanoic acid and lauric acid, which are known to contribute to penetration enhancement in topical skin delivery.

### BACKGROUND OF THE INVENTION

Visible skin damage, particularly on the face, is one of the most common targets of dermatological and cosmetic treatments. Untreated facial skin damage may cause anxiety and psycho-emotional trauma. Facial skin damage is often caused by photoaging, and may present itself as actinic keratosis, solar lentigines, loss of underlying skin collagen, loss of elastin, wrinkles and sagging skin. At an early stage, photoaging may present as inflamed erythema as well as enlarged freckles. Other facial skin damage may be caused by acne, resulting in both inflamed pores and post-acne scarring.

Topical retinoids are the first line treatment for skin damage cause by photoaging and acne in both cosmetic and prescription drug products. These include first generation retinoids that are related to Vitamin A, such as retinol (retinyl alcohol) and retinoic acid. Furthermore, there second generation retinoids that are further variations of Vitamin A. These materials are all lipophilic and relatively easy to formulate in topical products, though they suffer from two principal drawbacks: they cause significant erythema, scaling and peeling of skin, and they are teratogenic.

Trifarotene (4-[3-(3-tert-Butyl-4-pyrrolidin-1-ylphenyl)-4-(2-hydroxyethoxy)phenyl]benzoic acid) is a fourth-generation retinoid used topically to treat acne as a selective retinoic acid receptor gamma (RAR-gamma) agonist. However, derivatives with different chemical properties are needed in order to provide enhanced efficacy.

The present disclosure relates to trifarotene fatty acid esters. In certain embodiments, upon hydrolysis in the skin, the shorter chain esters release short chains fatty acids such as octanoic acid and lauric acid, which can provide antimicrobial benefits on the skin. Furthermore, shorter chains such as lauric acid and longer chains such oleic acid released upon ester hydrolysis are known to contribute to penetration enhancement in topical skin delivery.

### SUMMARY OF THE INVENTION

In various aspects, the present invention relates to compounds of Formula I:

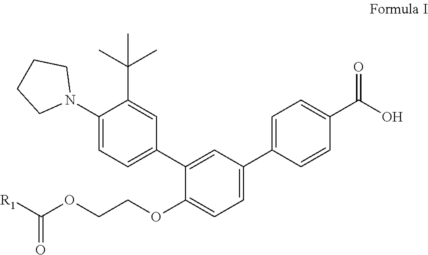

wherein R₁ is an unsubstituted hydrocarbyl having seven to seventeen carbon atoms.

Further aspects relate to topical skin care compositions comprising one or more compounds of Formula I in a cosmetically acceptable carrier.

Another aspect is directed to a method of inhibiting or reducing signs of photoaging or acne comprising applying a composition described herein topically to the skin of a subject in need thereof.

### DETAILED DESCRIPTION OF THE INVENTION

In general, the present invention relates to trifarotene fatty acid esters, various external compositions containing one or more trifarotene fatty acid esters, methods of using these compositions, and processes for preparing these compositions. The external compositions are applied to skin where upon hydrolysis in the skin, the shorter chain esters release short chain fatty acids such as octanoic acid and lauric acid, which can provide antimicrobial benefits on the skin. Furthermore, shorter chains such as lauric acid and longer chains such oleic acid released upon ester hydrolysis are known to contribute to penetration enhancement in topical skin delivery.

Accordingly, the present invention provides for active agents that are trifarotene derivatives that can be used for treating acne. These fourth generation retinoids include esters of C₈-C₁₈ fatty acids with trifarotene. For example, the trifarotene esters of fatty acids include those of Formula I:

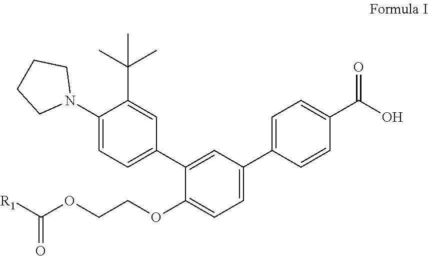

wherein R₁ is an unsubstituted hydrocarbyl having seven to seventeen carbon atoms. The hydrocarbyl can be branched or linear, saturated or unsaturated. In various embodiments, R₁ is a saturated or unsaturated, linear or branched, aliphatic hydrocarbyl group. Preferably, R₁ is an unsubstituted branched or linear alkyl or alkenyl having seven or more carbon atoms, such as a C₇-C₁₇ carbon chain. When R₁ is alkenyl, the substituent has at least one double bond, but can optionally have two or more double bonds. Examples of acceptable hydrocarbyl groups (R₁) include C₇-C₁₇ alkyl or alkenyl groups. Among them C₇, C₁₁, and C₁₅ alkyl and C₁₇ alkenyl are preferable. The alkyl or alkenyl ester chain in the compound of Formula I is preferably one that that is soluble in an oil phase and is mutually soluble with lipophilic emollients typically used in topical skin products. The compounds may be in the form of an amorphous solid (i.e. non-crystalline solid).

In certain embodiments, the compound is selected from the group consisting of:

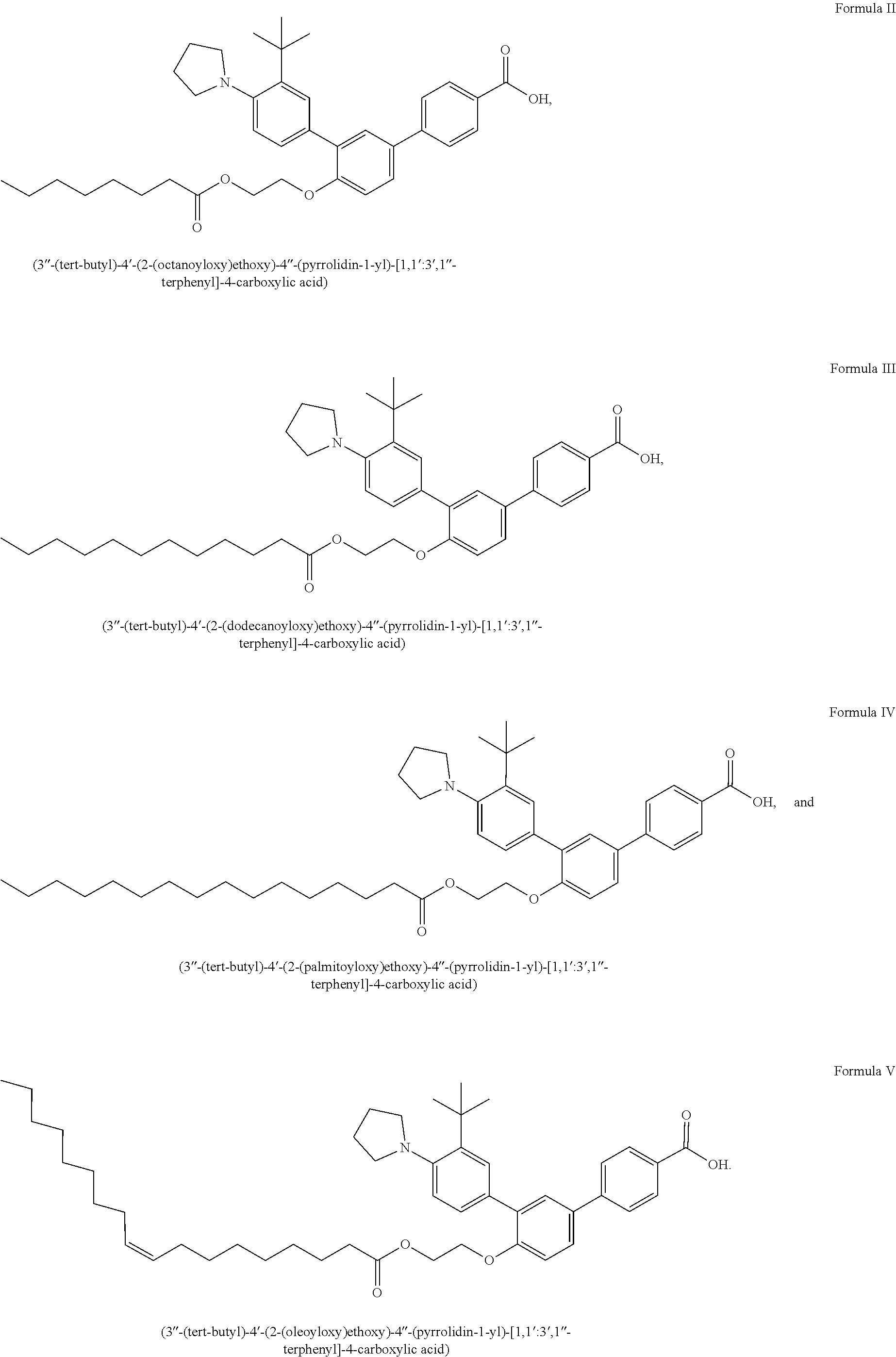 [SMILES: CCCCCCCC(=O)OCCOc1ccc(-c2ccc(C(=O)O)cc2)cc1-c1ccc(N2CCCC2)c(C(C)(C)C)c1 ; CCCCCCCCCCCC(=O)OCCOc1ccc(-c2ccc(C(=O)O)cc2)cc1-c1ccc(N2CCCC2)c(C(C)(C)C)c1 ; CCCCCCCCCCCCCCCC(=O)OCCOc1ccc(-c2ccc(C(=O)O)cc2)cc1-c1ccc(N2CCCC2)c(C(C)(C)C)c1 ; "and" ; CCCCCCCC/C=C\CCCCCCCC(=O)OCCOc1ccc(-c2ccc(C(=O)O)cc2)cc1-c1ccc(N2CCCC2)c(C(C)(C)C)c1]

Esterification of a fatty acid with trifarotene can be accomplished by the following processes and others known in the art. The trifarotene alcohol and fatty acid can be added in stoichiometric quantities, and esterification catalysts, such as sulfonic acids and organo-tin catalysts, can be used to increase the rate of reaction, while the water of reaction can be removed by a reflux condenser. Alternatively, acyl acid chloride (also called alkyl acid chloride) can used instead of the fatty acid and the reaction can proceed rapidly without the need for catalysts. For example, trifarotene can be reacted with octanoyl chloride in a suitable solvent to produce trifarotene octanoyl esters (trifarotene caprylate).

The present invention also comprises mixtures of the active agent in cosmetically acceptable carriers, such as oils and emollients (i.e., lipophilic carriers). These include but are not limited to emollient esters, triglycerides, vegetable oils, polyesters, polyol esters, wax esters such as jojoba oil, guerbet esters, fatty alcohol, fatty acid ethers, vegetable waxes, hydrocarbon fluids such as mineral oils, petrolatum, alkanes and isoalkanes, silicones, squalane and other topical lipophilic emollients known in the art. The cosmetically acceptable lipophilic carrier can comprise an ester. The lipophilic solutions include the active agent in an amount necessary to achieve anti-acne effects. Typically, the trifarotene fatty acid ester is soluble in the lipophilic carrier at a minimum of about 1% to 10% or more by weight. The trifarotene fatty acid ester can be soluble in the lipophilic carrier at a minimum of 5%, about 6%, about 7%, about 8%, about 9%, or about 10% or more by weight.

The trifarotene fatty acid ester can be formulated in a premix composition with a lipophilic carrier at even higher concentrations of the trifarotene fatty acid ester. Typically, the concentration of the trifarotene fatty acid ester in the premix composition will be at least about 1% by weight, and the weight ratio of trifarotene fatty acid ester to lipophilic carrier is from about 1:999 to about 2:8. The weight ratio of the compound to the cosmetically acceptable lipophilic carrier can be at least 1:99. The weight ratio of the compound to the cosmetically acceptable lipophilic carrier can be from about 1:9 to about 3:7. The premix composition may in the form of a homogeneous liquid solution or single phase amorphous solid.

The present invention further comprises other external compositions containing the active agent dissolved or dispersed in oils and emollients, typically referred to as an oil phase, where the oil phase is further dispersed in a physiologically acceptable topical formulation, such as water-in-oil or oil-in-water emulsion, or a suspension of oil droplets within aqueous matrix such as a gel. The aqueous phase of such compositions comprise water and may also include emulsifiers, glycerin and preservatives (e.g., caprylyl glyceryl ether, propanediol and combinations thereof).

In addition, the present invention further relates to external compositions containing a trifarotene fatty acid ester to inhibit or eliminate signs of photoaging and acne, such as reducing aging spots, discoloration spots, redness, blemishes, fine lines, and wrinkles. Said external compositions are particularly distinct because the shorter chain esters, upon hydrolysis in the skin, release short chains fatty acids such as octanoic acid and lauric acid, which are known have antimicrobial activity and known to contribute to penetration enhancement in topical skin delivery. Typically, the trifarotene fatty acid ester active agent is present in such external compositions at a concentration of from about 10 ppm to about 1000 ppm and can be readily formulated from the premix composition described above by mixing the premix composition with other cosmetically acceptable ingredients.

The trifarotene fatty acid ester herein may be combined in an external composition with other ingredients intended to inhibit photoaging and acne, or to reduce aging spots, discoloration spots, redness, blemishes, fine lines, and wrinkles. Examples of said ingredients include, but are not restricted to ascorbic acid and derivatives thereof, ferulic acid, azelaic acid, kojic acid, mandelic acid, alpha-hydroxy acids, beta-hydroxy acids, lipohydroxyacids, fruit acids, polysaccharides, hyaluronic acid, humectants, gluconolactone, heparan sulfate, arbutin, niacinamide, resveratrol, hydroquinone, bakuchiol, exfoliants, keratolytics, plant extracts, marine extracts, ferment extracts, isoflavones, bisabolol, anti-aging peptides, retinol, retinoic acid, retinyl esters, retinoic acid esters, adapalene, adapalene esters, and other retinoids.

The compositions of trifarotene fatty acid esters and one or more additional ingredients described herein can be used as topical skin care compositions. The present disclosure is further directed to methods of inhibiting or reducing signs of photoaging or acne comprising applying any of the compositions described elsewhere herein topically to the skin of a subject in need thereof.

### Definitions

The term “fatty acid,” as used herein, refers to a carboxylic acid consisting of an unsubstituted, saturated or unsaturated hydrocarbyl and a terminal carboxyl group. Unless otherwise specified, a fatty acid preferably contains 2 to about 30 carbon atoms or about 8 to about 18 carbon atoms, including branched or linear species. Fatty acids can have an even or odd number of carbon atoms but preferably have an even number of carbon atoms.

As used herein, the term “hydrocarbyl” refers to hydrocarbyl moieties, unless otherwise specified, preferably containing, 1 to about 50 carbon atoms, preferably 1 to about 30 carbon atoms, and even more preferably 1 to about 18 carbon atoms, including branched or unbranched, and saturated or unsaturated species. Hydrocarbyl groups can be aromatic or aliphatic (i.e. non-aromatic). Unsubstituted hydrocarbyl groups can be selected from the group consisting of alkyl, alkenyl, cycloalkyl, cycloalkylalkyl, aryl, aralkyl, and the like. Unsubstituted hydrocarbyl groups contain only carbon and hydrogen atoms and do not contain further functional substituent groups. A hydrocarbyl may be optionally substituted hydrocarbyl. Hence, various substituted hydrocarbyls can be further selected from substituted alkyl (e.g., cyano, alkoxy, alkylamino, thioalkyl, haloalkyl), substituted cycloalkyl (e.g., heterocyclyl, N-heterocyclyl, heterocyclylalkyl), substituted aryl (i.e. heteroaryl, N-heteroaryl, heteroarylalkyl), and the like.

The term “alkenyl,” as used herein, refers to a straight or branched hydrocarbon radical, preferably having 2, 3, 4, 5, 6, 7, 8, 9, 10, 11, 12, 13, 14, 15, 16, 17, 18, 19, 20, 21, 22, 23, 24, 25, 26, 27, 28, 39, 30, 31, or 32 carbons, and having one or more carbon-carbon double bonds. Alkenyl groups include, but are not limited to, ethenyl, 1-propenyl, 2-propenyl (allyl), iso-propenyl, 2-methyl-1-propenyl, 1-butenyl, and 2-butenyl. Alkenyl groups can be unsubstituted or substituted by one or more suitable substituents, as defined above.

The term “alkyl,” as used herein, refers to a linear or branched hydrocarbon radical, preferably having 1 to 32 carbon atoms (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 39, 30, 31, or 32 carbons). Alkyl groups include, but are not limited to, methyl, ethyl, n-propyl, isopropyl, n-butyl, iso-butyl, secondary-butyl, and tertiary-butyl. Alkyl groups can be unsubstituted or substituted by one or more suitable substituents, as defined above.

The term “substituted” as used herein, refers to chemically acceptable functional substituent groups, preferably moieties that does not negate the activity of the compounds. Such substituents include, but are not limited to hydroxy groups, oxo groups, alkoxy groups, and/or cycloalkyl groups.

The term “alkoxy,” as used herein, refers to an alkyl group, as defined herein, appended to the parent molecular moiety through an oxygen atom.

The term “aryl,” as used herein, means monocyclic, bicyclic, or tricyclic aromatic radicals such as phenyl, naphthyl, tetrahydronaphthyl, indanyl and the like; optionally substituted by one or more suitable substituents, preferably 1 to 5 suitable substituents, as defined above.

The term “cycloalkyl,” as used herein, refers to a mono, bicyclic or tricyclic carbocyclic radical (e.g., cyclopropyl, cyclobutyl, cyclopentyl, cyclohexyl, cycloheptyl, cyclooctyl, cyclononyl, cyclopentenyl, cyclohexenyl, bicyclo[2.2.1]heptanyl, bicyclo[3.2.1]octanyl and bicyclo[5.2.0]nonanyl, etc.); optionally containing 1 or 2 double bonds. Cycloalkyl groups can be unsubstituted or substituted by one or more suitable substituents, preferably 1 to 5 suitable substituents, as defined above.

The term “hydroxy,” as used herein, refers to an —OH group.

The term “oxo,” as used herein, refers to a double bonded oxygen (═O) radical wherein the bond partner is a carbon atom. Such a radical can also be thought as a carbonyl group.

Having described the invention in detail, it will be apparent that modifications and variations are possible without departing from the scope of the invention defined in the appended claims.

### EXAMPLES

The following non-limiting examples are provided to further illustrate the present invention.

### Example 1—Synthesis of Trifarotene Fatty Acid Ester Active Agent

Trifarotene caprylate ester was prepared according to the following steps:

1. A 250 ml flask was charged with a mechanical stirrer and a dropping funnel.

2. 10 g of trifarotene, 5.2 g of triethylamine and 100 ml of dichloromethane was added to the flask.

3. The flask was placed in an ice/water bath. The stirrer was turned on to agitate the mixture for 1 hour.

4. At 0° C., 4.3 g of octanoyl chloride was added dropwise into the flask.

5. The reaction mixture was slowly warmed to room temperature and kept stirring.

6. The reaction was monitored by TLC. When the reaction was done, water was added to quench the reaction.

7. The reaction mixture was washed with water 3 times and dried with Na₂SO₄, followed by column purification, to produce 16 g of final product.

### Example 2—Trifarotene Ester in Solution

A premix solution of 1% trifarotene caprylate was made by mixing 1 gram of trifarotene caprylate with 99 grams of the emollient ester caprylic/capric triglyceride, heating to 75° C. for ten minutes and cooling to room temperature. It formed a homogeneous clear solution.

### Example 3—Trifarotene Caprylate Lotion External Composition

TABLE 1

| Phase | Ingredient | % wt/wt |
|---|---|---|
| A | DI water | 80.0 |
| | Emulsifier Blend (Pisum Sativum Peptide, | 3.0 |
| | Sodium Stearoyl Lactylate, Xanthan Gum, | |
| | Sodium Citrate, Magnesium Stearate) | |
| | Glycerin | 5.0 |
| | Diol Preservative Blend | 2.0 |
| B | Trifarotene Caprylate and Caprylyl/Capric | 1.0 |
| | Triglyceride 1% premix solution from | |
| | Example 2 | |
| | Sunflower Oil | 4.0 |
| | Squalane | 5.0 |
| C | Citric Acid | QS pH 5.5 |

Lotion was produced by first combining Phase A, mixing until uniform and clump free, and heating to 80-85° C. Then Phase B was combined and mixed until uniform at 80-85° C. Finally, Phase B was added into Phase A at the same temperature and mixed until uniform, cooling to room temperature and adjusting pH using Phase C. The final formula contained 100 ppm of trifarotene caprylate.

### Example 4—Trifarotene Caprylate Composition Clinical Test

The lotion of Example 3 was tested on adult human subjects for purposes of evaluating anti-aging and skin rejuvenation efficacy.

Ten female subjects ages 35-65 (mean age 57) were enrolled in the study by Validated Claim Support LLC of Teaneck, NJ. They were provided the lotion of Example 3 and instructed to apply it once per day at bedtime to their facial skin as they would normally do in their beauty routine.

A CUTOMETER device (available from Courage & Khazaka Electronic GmbH) was used to periodically measure skin firmness and elasticity. After twelve weeks, 50% of the participants had increased skin firmness, and 70% of the participants has increased skin elasticity.

Each participant's facial skin was also periodically graded by a trained clinical technician. The grading system tracked changes in skin condition over the twelve-week study, and the results are reported in Table 2.

TABLE 2

| | Improvement | Improvement |
|---|---|---|
| Expert Grading Assessment | after 8 Weeks | after 12 Weeks |
| Texture/Smoothness | 18.52% | 29.63% |
| Fine Lines/Wrinkles | 8.93% | 8.93% |
| Evenness of Skin Tone | 10.91% | 21.82% |
| Clarity | 25.86% | 34.48% |
| Overall Appearance | 15.25% | 27.12% |

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions, methods, and processes without departing from the scope of the invention, it is intended that all matter contained in the above description shall be interpreted as illustrative and not in a limiting sense.

What is claimed is:

1. A compound of Formula I: 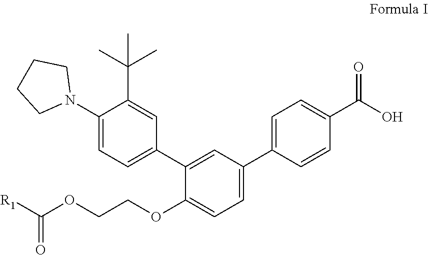
wherein R₁ is an unsubstituted hydrocarbyl having seven to seventeen carbon atoms.

2. The compound of claim 1, wherein R₁ is saturated or unsaturated, linear or branched, aliphatic hydrocarbyl group.

3. The compound of claim 1, wherein R₁ is an unsubstituted linear or branched alkyl or alkenyl.

4. The compound of claim 1, wherein R₁ is C₇, C₁₁, or C₁₅ alkyl.

5. The compound of claim 1, wherein R₁ is C₁₇ alkenyl.

6. The compound of claim 1, wherein the compound is selected from the group consisting of:

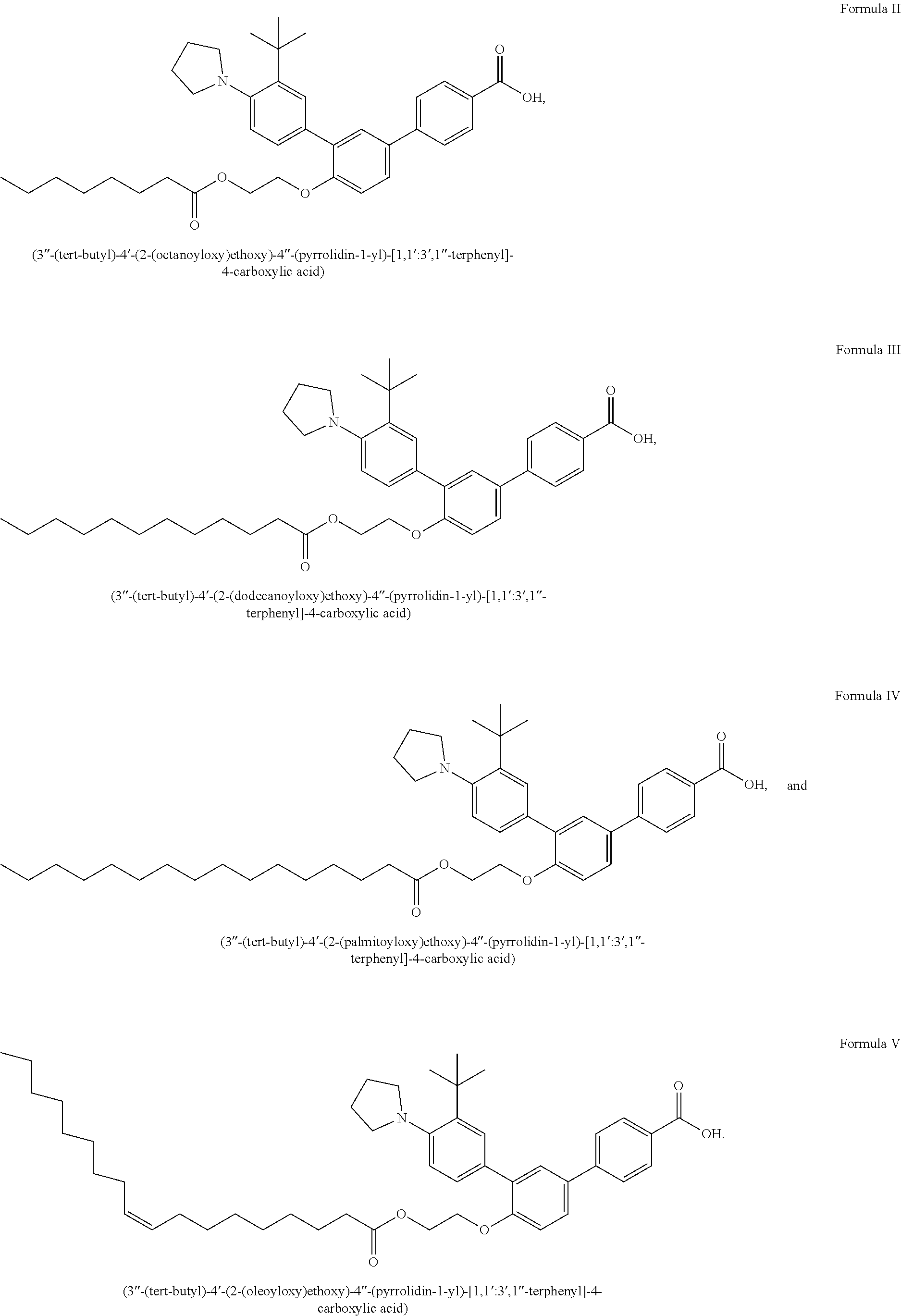 [SMILES: CCCCCCCC(=O)OCCOc1ccc(-c2ccc(C(=O)O)cc2)cc1-c1ccc(N2CCCC2)c(C(C)(C)C)c1 ; CCCCCCCCCCCC(=O)OCCOc1ccc(-c2ccc(C(=O)O)cc2)cc1-c1ccc(N2CCCC2)c(C(C)(C)C)c1 ; CCCCCCCCCCCCCCCC(=O)OCCOc1ccc(-c2ccc(C(=O)O)cc2)cc1-c1ccc(N2CCCC2)c(C(C)(C)C)c1 ; CCCCCCCC/C=C\CCCCCCCC(=O)OCCOc1ccc(-c2ccc(C(=O)O)cc2)cc1-c1ccc(N2CCCC2)c(C(C)(C)C)c1]

7. A topical skin care composition comprising the compound of claim 1 in a cosmetically acceptable lipophilic carrier, wherein the compound is at least partially soluble in the cosmetically acceptable lipophilic carrier.

8. The composition of claim 7, wherein the cosmetically acceptable lipophilic carrier is selected from the group consisting of emollient esters, triglycerides, vegetable oils, polyesters, polyol esters, wax esters, guerbet esters, fatty alcohols, fatty acid ethers, vegetable waxes, hydrocarbon fluids, petrolatum, alkanes, isoalkanes, silicones, squalane and combinations thereof.

9. The composition of claim 7, wherein the cosmetically acceptable lipophilic carrier comprises an ester.

10. The composition of claim 7, wherein the compound is soluble in the cosmetically acceptable lipophilic carrier at a minimum of about 1% by weight.

11. The composition of claim 7, wherein the weight ratio of the compound to the cosmetically acceptable lipophilic carrier is at least 1:99.

12. The composition of claim 7, wherein the weight ratio of the compound to the cosmetically acceptable lipophilic carrier is from about 1:9 to about 3:7.

13. The composition of claim 7, wherein the composition comprises an oil phase comprising the compound in the lipophilic carrier and an aqueous phase comprising water.

14. The composition of claim 13 in the form of water-in-oil emulsion, an oil-in-water emulsion, or a suspension of oil droplets within aqueous matrix such as a gel.

15. The composition of claim 7, wherein the composition further comprises at least one ingredient selected from the group consisting of ascorbic acid and derivatives thereof, ferulic acid, azelaic acid, kojic acid, mandelic acid, alpha-hydroxy acids, beta-hydroxy acids, lipohydroxyacids, bakuchiol, fruit acids, polysaccharides, hyaluronic acid, humectants, gluconolactone, heparan sulfate, arbutin, niacinamide, resveratrol, hydroquinone, exfoliants, keratolytics, plant extracts, marine extracts, ferment extracts, isoflavones, bisabolol, anti-aging peptides, retinol, retinoic acid, retinyl esters, retinoic acid esters, adapalene, adapalene esters, and other retinoids.

16. A method of inhibiting or reducing signs of photoaging or acne comprising applying the composition of claim 7 topically to the skin of a subject in need thereof.
